(19) United States
(12) Patent Application Publication
(10) Pub. No.: US 2022/0133967 A1
(43) Pub. Date: May 5, 2022

(54) HEMODIALYZER

(71) Applicant: Fresenius Medical Care Holdings, Inc., Waltham, MA (US)

(72) Inventors: Hui SHAO, Salt Lake City, UT (US); Jiunn TEO, Pleasant View, UT (US); Chih-Hu HO, Farmington, UT (US)

(73) Assignee: Fresenius Medical Care Holdings, Inc., Waltham, MA (US)

(21) Appl. No.: 17/512,704

(22) Filed: Oct. 28, 2021

Related U.S. Application Data
(60) Provisional application No. 63/107,566, filed on Oct. 30, 2020.

(51) Int. Cl. A61M 1/16; B01D 69/08; B01D 71/32; B01D 71/02; B01D 71/44

(52) U.S. Cl. CPC A61M 1/16; B01D 69/087; B01D 71/32; A61M 2207/00; B01D 71/44; A61M 2205/7527; A61M 2205/3334; B01D 71/024

(57) ABSTRACT

A hollow fiber membrane and methods of making the hollow fiber membrane are described. The membrane includes a hydrophobic polymer such as polysulfone, a hydrophilic polymer such as polyvinylpyrrolidone (PVP), and a fluropolymer additive, and optionally a stabilizer, for instance, to stabilize the fluoropolymer additive in the membrane, particularly during conditioning or E-beam sterilization or both. Further conditioning improvements to membrane manufacturing are disclosed. The membrane may be incorporated into a dialysis filter for use in hemodialysis and related applications. The membrane has improved hemocompatibility, charge stability, or middle molecule clearance compared to conventional membranes. Also disclosed is a method of evaluating membrane charge stability.

This application claims the benefit under 35 U.S.C. § 119(e) of prior U.S. Provisional Patent Application No. 63/107,566, filed Oct. 30, 2020, which is incorporated in its entirety by reference herein.

### FIELD

The present invention, in part, relates to a method of making hollow fiber membranes, for instance, for use in treating blood. Preferably, the hollow fiber membranes have improved chemical stability, and/or improved hemocompatibility, and/or improved performance as compared to conventional membranes. The present invention further relates to methods of making dialysis filters comprising the membranes, and methods of using the dialysis filters.

### BACKGROUND

Dialysis is commonly used to treat patients suffering from end stage renal disease (ESRD). Various unwanted substances can be removed from a patient's blood during a dialysis session. These include metabolic waste products (e.g., urea, creatinine, middle molecular weight proteins), other toxins, and excess fluid. In hemodialysis (HD), blood is withdrawn from a patient and passed through a dialysis filter containing thousands of thin, porous, semipermeable and elongated hollow fiber membranes anchored into a potting compound at both ends of the filter. The blood is channeled through the internal luminal space of the membranes (the “blood compartment”), exchanging solutes and water via mostly diffusive processes with a dialysate solution flowing in the space outside the fibers but within the housing of the filter (the “dialysate compartment”) in a direction countercurrent to the blood. Variations of HD include hemodiafiltration (HDF) and hemofiltration (HF), which employ pressure gradients in the filter to further drive convective flow of solutes and water out of blood.

Aside from a small surface area in the blood tubing, the primary surface that makes direct contact with blood in a dialysis circuit is the inner surface of these hollow fiber membranes. A common challenge of working with blood is undesired coagulation, which is promoted by activation of inflammatory and coagulation factors as the blood contacts the artificial surfaces of medical devices. Anticoagulation (e.g., heparin) therapy is widely prescribed to dialysis patients to minimize extracorporeal clotting. However, heparin therapy is costly, not universally tolerated by dialysis patients, and is associated with numerous side effects. Thus, a goal of modern renal replacement therapy is to improve hemocompatibility and reduce or eliminate heparin demand.

U.S. Patent Pub. 2011/0009799 relates to antithrombogenic extracorporeal blood circuits and components thereof, such as hollow fiber membranes, blood tubing, and filters, as well as their use in hemodialysis, hemofiltration, hemodiafiltration, hemoconcentration, blood oxygenation, and related applications. The hollow fiber membrane includes a fluoropolymer additive serving as a surface modifying macromolecule (SMM). The SMM-modified filter has a lower average header pressure and thrombogenicity than a control filter in heparinized blood tests.

Further improvements in dialysis manufacturing are needed to more successfully and stably integrate SMMs into dialysis membranes while maintaining or improving hemocompatibility and performance of the dialyzer.

### OBJECT OF THE INVENTION

A hollow fiber membrane with excellent hemocompatibility is needed to reduce or eliminate therapeutic anticoagulant needs in hemodialysis patients. Improvements in the manufacture of a membrane incorporating one or more fluoropolymer additives should provide increased membrane stability and improved middle molecule clearance with only minimal loss of albumin, as compared to conventional membranes. Other objects and advantages are described herein.

### SUMMARY OF THE INVENTION

A feature of the present invention is to provide a method for making membranes and/or compositions containing surface modifying macromolecules which meet the above and/or other needs.

Additional features and advantages of the present invention will be set forth in part in the description that follows, and in part will be apparent from the description, or may be learned by practice of the present invention. The objectives and other advantages of the present invention will be realized and attained by means of the elements and combinations particularly pointed out in the description and appended claims.

To achieve these and other advantages, and in accordance with the purposes of the present invention, as embodied and broadly described herein, the present invention relates to a hollow fiber membrane for blood purification or other uses. The hollow fiber membrane includes at least one hydrophobic base polymer; at least one hydrophilic polymer; at least one fluoropolymer additive; and optionally at least one stabilizer, wherein a fluorine content on an inner surface of the hollow fiber membrane is, for instance, from 5 to 10 atomic % (F), as determined by X-ray photoelectron spectrometry (XPS).

The present invention further relates to a dialysis filter for use in hemodialysis. The dialysis filter includes the hollow fiber hollow fiber membrane of present invention.

The present invention also relates to a method of manufacturing the dialysis filter. The method includes the steps of:

A) preparing a spin mass comprising an aprotic solvent, the hydrophobic base polymer, the hydrophilic polymer, and the fluropolymer additive in a concentration of from 0.9% to 1.3% w/w, based on the total weight of the spin mass;

(B) extruding said spin mass from an outer annular orifice through a tube in-orifice spinneret into an aqueous solution to form the hollow fiber membrane, and

(C) isolating the hollow fiber membrane,

wherein the dialysis filter has a beta-2-microglobulin (B2M) reduction ratio greater than 60%; a β₂-microglobulin (B2M) clearance of at least 65 ml/min per membrane area of 1.5 m² at a blood flow rate of 300 ml/min, a dialysate flow rate of 500 ml/min, and an ultrafiltration rate of 0.0 ml/min; and an albumin sieving coefficient of less than 0.01, when operated in a hemodialysis mode.

In addition, the present invention relates to a method of hemodialysis. The method includes passing blood through a first chamber of a dialysis filter of present invention such that the blood contacts a first side of the hollow fiber membrane of the present invention; and passing a dialysis solution through a second chamber of the dialysis filter such that the dialysis solution contacts a second opposite side of the membrane to remove waste products from the blood, wherein the first chamber is inside the hollow fiber membrane and the second chamber is between the hollow fiber membrane (outer wall) and an inner wall of the dialysis filter.

It is to be understood that both the foregoing general description and the following detailed description are exemplary and explanatory only and intended to provide a further explanation of the present invention, as claimed.

### BRIEF DESCRIPTION OF THE FIGURES

The present invention at times is described with reference to the accompanying drawings, in which several exemplary embodiments are shown. The subject matter of the present invention, however, may be embodied in many different forms and should not be construed as limited to these specific embodiments. In the drawings, like numbers refer to like elements throughout.

FIG. 1 is a general schematic of the chemical structure of the SMM1 molecule.

FIGS. 2A and 2B are cross-sectional Scanning Electron Microscopy (SEM) images of the: (2A) SMM1 modified membrane; and (2B) Standard PSF membrane.

FIG. 3 is a plot showing zeta potential measurements as a function of pH in several tested dialyzers (zeta potential vs pH).

FIG. 4 is a plot showing measurements of the blood clotting time with an in vitro coagulation test model in the Standard PSF dialyzer (left) and the SMM1-modified dialyzer (right).

FIG. 5 is a plot showing platelet count reduction in the Standard PSF dialyzer (upper trace, circles) and SMM1-modified dialyzer (lower trace, squares).

FIG. 6 is a plot showing measurements of cellular activation factor Platelet Factor 4 (PF-4) in the Standard PSF dialyzer (upper trace, circles) and the SMM1-modified dialyzer (lower trace, squares).

FIG. 7 is a plot showing mean serum albumin levels in a study of the SMM1-modified dialyzer. In visit 13, the left block is “pre-HD” and the right block is “post-HD”. For all of the other visits (visits 22, 34, and 46), all of the blocks for these visits are “pre-HD” only.

FIG. 8 is a plot showing Beta-2-microglobulin removal rate (%) in a clinical study of the SMM1-modified dialyzer.

### DETAILED DESCRIPTION

Hemodialysis (HD) is the most common renal replacement therapy in patients suffering from acute kidney injury (AKI) or end stage renal disease (ESRD). Blood passes through thousands of hollow fibers in the HD filter, allowing toxins and fluids to pass across the semi-permeable membrane walls of the fibers and to be removed from the body.

Hemodialysis is also associated with several complications, however. Contact of blood with the artificial surfaces of the extracorporeal circuit can activate the coagulation cascade. This results in thrombosis and clotting inside the hollow fibers and bloodlines, rendering the circuit unusable for continuation of treatment and preventing the return of blood to the patient, resulting in blood loss. Anticoagulants are used to prevent this clotting and preserve adequate blood flow inside the hemodialyzers. While several anticoagulants have been used over the decades, heparin is the most common agent. A plethora of side effects for heparin have been reported, however, including heparin induced-thrombocytopenia, necrosis, hypersensitivity reactions, hemorrhage, hyperkalemia, alopecia, bone loss, and osteoporosis.

In an effort to reduce the need for heparin during dialysis and minimize the complications associated with systemic heparin administration, continuing efforts have been made to improve membrane hemocompatibility. A major effort is directed at modifying the blood-contacting surface of the membrane. Early approaches utilizing heparin-coated surfaces showed some success, but some patients still experienced heparin-related side effects. An alternative approach adds surface modifying molecules (SMMs) directly into membrane-forming compositions during the production process. This simplifies manufacturing by eliminating the additional coating step.

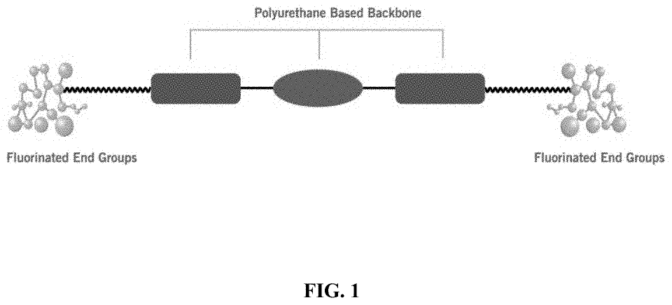

ENDEXO (Interface Biologics, Inc., Toronto, Ontario) is a family of SMMs that can be admixed into the membrane spinning solution at 0.005% to 10% (w/w). One member of this family, SMM1, is a low molecular weight fluoropolymer additive that spontaneously migrates to the surface of polysulfone-based hollow fiber membranes to provide passive surface modification. SMM1 is illustrated schematically in FIG. 1. SMM1 consists of a polyurethane base polymer which is synthesized from 1,6-hexanediisocyanate (HDI, rectangles) and polypropylene glycol (oxide) (PPG or PPO, ovals). The polyurethane base polymer is end-capped with active functionalized fluorinated segments, 1H, 1H, 2H, 2H Perfluoro-1-octanol (PFO). The molecular weight of SMM1 is ˜10 kDa relative to polystyrene reference standards. In the presence of blood, the modified membrane surface is shown to suppress procoagulant protein conformation, reduce platelet adhesion and inhibit platelet activation. ENDEXO has been approved for use in peripherally inserted central catheters in the United States. U.S. Patent Pub. 2011/0009799 discloses a generalized scheme for manufacturing a polysulfone-based dialysis membrane integrating SMMs.

SMM1 is not a coating on the membrane but is rather blended with the polymers, such as polysulfone and polyvinylpyrollidone (PVP), during fiber formation. This blending strategy allows SMM1 to become part of the blood contacting interface and potentially create a more neutral surface. SMM1 is hydrophobic, likely due to the terminal fluorinated end groups, leading to poor van der Waals interactions with water. The standard polysulfone-based membrane is hydrophilic due to the presence of PVP, but with the addition of the hydrophobic SMM1, the modified membrane is expected to be more hydrophobic than a standard polysulfone-based membrane.

The present invention describes further process improvements to membrane and filter manufacturing that permit more effective integration of one or more fluoropolymer additives (e.g., SMM1) into hollow fiber membranes. The disclosed hollow fiber membrane is associated with one or more benefits in hemodialysis when compared to conventional hemodialysis, including but not limited to: reduced need for heparin; and/or comparable or superior performance (e.g., urea, creatinine, Kuf); and/or improved middle molecule clearance (i.e., middle molecular weight proteins) while still only having minimal albumin loss; and/or improved hemocompatibility; improved membrane stability (surface charge/zeta potential); and/or improved incorporation of SMM and PVP into the membrane (XPS, contact angle, Raman spectroscopy); and/or reduced leachability of PVP.

These findings demonstrate that the inventive fluoropolymer-containing dialyzer can significantly improve patient outcomes while reducing the need for heparin long term.

According to one aspect, the present invention thus relates to a hollow fiber membrane formed from one or more hydrophobic polymers (e.g., one or more hydrophobic-base polymers), one or more hydrophilic polymers, and one or more fluoropolymer additives. The hollow fiber membrane preferably has improved hemocompatibility and/or chemical stability when exposed to blood compared to conventional hollow fiber membranes. In embodiments, the hollow fiber membrane can be formed from a spin mass (i.e., spinning solution) comprising at least one hydrophobic polymer, at least one hydrophilic polymer, and at least one fluoropolymer (or fluoropolymer additive), and an aprotic solvent.

Hydrophobic polymers have been widely used as polymeric materials in hollow fiber membranes. In particular, polysulfones are synthetic hydrophobic polymers that are widely used in hollow fiber membranes for dialysis due to their excellent fiber spinning properties and biocompatibility. Therefore, in some embodiments the spinning solution used in the instant invention comprises at least one polysulfone.

The term “polysulfone” is used herein as a general term for polymers comprising units of a polymeric aryl sulfone. Thus, the term encompasses a polysulfone made from bisphenol A (PSF), a polyethersulfone (PES), polysulfone made from bisphenol S, poly(aryl)ethersulfone (PAES), and copolymers made therefrom. Polysulfone based polymers show in general a good hemocompatibility when used as dialysis filter membranes. It has also been found that polysulfones exhibit a good chemical compatibility with fluoropolymer additives acting as surface modifying molecules, yielding membranes with high mechanical strength.

In a preferred embodiment, the proportion of the polysulfone in the spin mass is from 10-20 wt %, preferably from 15-20 wt %, and more preferably about 16 wt %, based on total weight of the spin mass. In preferred embodiments, the polysulfone is PSF.

However, pure hydrophobic PSF cannot be used directly for some applications (e.g., dialysis membranes) because PSF decreases the wetting characteristics of the membrane in an aqueous environment and negatively affects the clearance of toxins. To address this problem, a hydrophilic polymer such as polyvinylpyrrolidone (PVP) or polyethylene glycol is typically added to PSF, rendering at least a part of the membrane surface hydrophilic. A hydrophilic polymer enhances hemocompatibility and helps wetting of the pore, which in turn enhances clearance of certain solutes from blood. Thus, in embodiments, the spinning mass preferably comprises a polyvinylpyrrolidone. The term “polyvinylpyrrolidone” comprises the homopolymer as well as co-polymers like vinylpyrrolidone-vinylacetate-based copolymers. Other suitable compounds are known in the art. In a preferred embodiment, the proportion of the PVP in the spin mass is 2-10 wt %, preferably 4-8 wt %, and more preferably 4-5 wt %, based on total weight of the spin mass.

In embodiments, the spin mass further comprises an aprotic solvent that can be dimethylformamide (DMF), dimethylsulfoxide (DMSO), diemethylacetamide (DMAC), or N-methylpyrrolidone (NMP) or a mixture of two or more thereof. These solvents are well suited to the production of membranes comprising a fluoropolymer additive such as SMM1. The proportions of the solvent can be adjusted to provide the desired solubility of the fluoropolymer, hydrophobic base polymer, and hydrophilic polymer, while also influencing membrane characteristics and performance.

Unless stated otherwise, references to a “Standard PSF membrane”, a “conventional membrane” or a “conventional PSF membrane” herein means the OPTIFLUX membrane (Fresenius Medical Care, Waltham, Mass., USA), for example, the membrane of the OPTIFLUX F160NR dialyzer, or a comparable membrane in the industry.

The spin mass further comprises a fluoropolymer additive. The fluoropolymer additive can be a surface-modifying macromolecule. The surface-modifying macromolecule can have the formula:

F_{T}-[B-(oligo)]n-B-F_{T},

wherein each B comprises a urethane; oligo comprises polypropylene oxide, polyethylene oxide or polytetramethylene oxide; each F_{T} is a polyfluoroorgano group; and n is an integer from 1 to 10. Preferably, the SMM has no Si moieties or siloxane groups present. Each B and each F_{T} can be the same or different.

Such a molecule is easily incorporated into the spin mass providing the desired effect of improving the anti-thrombotic properties of the membrane. This range of surface modifying molecules yields a balanced property of hydrophilicity and hydrophobicity. Such a suitable surface-modifying macromolecule is preferably made from 1H,1H,2H,2H-perfluorooctanol as F_{T}, hexamethylene diisocyanate as B, and propylene oxide as oligo.

In embodiments, the fluoropolymer additive is preferably SMM1. Other fluoropolymer additives having similar properties can additionally or alternatively be used herein. The spin mass in general can contain from 0.4 wt % to 1.9 wt % or more of one ore more fluoropolymer additives, based on total weight of the spin mass. In embodiments, the spin mass comprises between 0.4 wt % and 1.9 wt % SMM1, preferably between 0.8 wt % and 1.6 wt %, and more preferably between 0.9 wt % and 1.3 wt %, based on total weight of the spin mass.

The concentration of SMM1 or other fluoropolymer additive can also be expressed as a weight percentage with respect to the amount of the hydrophobic base polymer (e.g., PSF). Thus, in some embodiments, the concentration of SMM1 used in the spin mass, with respect to PSF, is between 4 wt % and 12 wt %, preferably between 5 wt % and 10 wt %, more preferably between 6 wt % and 8 wt %. When incorporated into a hollow fiber membrane manufactured according to the present invention, SMM1 migrates effectively to the inner surface/blood contacting surface of the forming hollow fiber membrane, stabilizing PVP in the membrane and improving hemocompatibility and performance.

The amount of the fluoropolymer additive, such as SMM1, present on the inner membrane surface can be estimated using various techniques known to the art, such as X-ray photoelectron spectrometry (XPS), which measures elemental atomic percentages of target elements (e.g., fluorine) on a surface. In some embodiments, the inner lumen surface of the hollow fiber membrane comprises at least 3 atomic % F, at least 4 atomic % F at least 5 atomic % F, at least 6 atomic % F, at least 7 atomic % F, at least 8 atomic % F, at least 9 atomic % F, or at least 10 atomic % F, as characterized by XPS (F) measurements. Due to the bonding with the hydrophilic polymer, more of the hydrophilic polymer is incorporated in the inner lumen compared with OPTIFLUX membrane. For instance, due to the bonding of the SMM1 bonds with PVP, more PVP is incorporated in the inner lumen compared with OPTIFLUX membrane. Therefore, the inner lumen of the membrane remains in the hydrophilic range, which allow to remove uremic toxins and extra waste water.

According to another aspect, the present invention relates to a dialysis filter incorporating the disclosed hollow fiber membranes. In preferred embodiments, the dialysis filter is a hemodialysis filter.

The term “dialysis filter” as used herein encompasses a filter housing comprising hollow fiber membranes in the form of a hollow fiber membrane bundle, the dialysis filter being configured for use in a dialysis machine which can be used by patients suffering from impaired kidney function. The bundle contains thousands (e.g., 3,000 to 30,000, typically around 10,000 to 20,000, more typically around 10,000 to 13,000) of individual hollow fiber membranes. Typically, the fibers are fine and of capillary size which typically ranges from about 150 to about 300 microns internal diameter with a wall thickness in the range of about 20 to about 50 microns.

The OPTIFLUX ENEXA ADVANCE FRESENIUS POLYSULFONE dialyzer, also referred to herein as the “ENEXA dialyzer” or the “SMM1-modified dialyzer”, is intended for patients with acute kidney injury or chronic renal disease when conservative therapy is judged to be inadequate. The ENEXA dialyzer is based off the widely-used OPTIFLUX family of high flux, E-beam sterilized, single-use dialyzers. In embodiments, the ENEXA dialyzer has comparable clearance performance (e.g., urea, creatinine, phosphate, Vitamin B12) to ENEXA dialyzers of similar size under similar conditions, and comparable albumin sieving coefficients (i.e., less than 0.01).

According to another aspect, the present invention relates to a method for manufacturing a hollow fiber membrane, the method comprising at least steps (A) to (D):

(A) preparing a spin mass or spinning solution comprising an aprotic solvent, a hydrophobic base polymer, a hydrophilic polymer, and a fluoropolymer additive, such as a fluoropolymer-based surface modifying molecule (SMM), wherein the spinning solution is heated to a temperature of 65-80° C.;

(B) extruding the spin mass or spinning solution from an outer annular orifice through a tube-in-orifice spinneret into an aqueous solution, with a centrally-controlled precipitation fluid consisting of a mixture of DMAC and water;

(C) isolating the formed hollow fiber membrane; and

(D) conditioning the hollow fiber membrane by exposure to saturated steam, rinsing with water, and air drying prior to sterilization.

In some embodiments, the spinning solution is heated to 75-80° C. prior to extrusion.

In embodiments, the centrally-controlled precipitation fluid consists of 50% DMAC and 50% water by weight.

In embodiments, the temperature of the annular spinneret is maintained at 35-45° C. during extrusion. In a preferred embodiment, the temperature of the annular spinneret is maintained at 38-42° C. during extrusion.

In embodiments, the extruded strand is guided through a precipitation gap of 200-600 mm prior to introduction into the precipitation bath. In a preferred embodiment, the precipitation gap is about 600 mm.

Without being bound by theory, it is believed that specific intra- and post-production conditioning steps, sterilization processing, and additional post-production treatment of dialysis fibers is associated with improved integration of the fluoropolymer additive (e.g., SMMs) and retention of hydrophilic polymer (e.g., PVP) in the membrane and other benefits disclosed herein.

As disclosed herein, conditioning involves passing the formed hollow fiber from the coagulation bath through a controlled sequence of steaming, rinsing, and drying. These conditioning steps are aimed at redistributing the active components, including the hydrophilic polymer and fluoropolymer additive (e.g., PVP and SMM1), on the surface of the membrane.

In some embodiments, the spin mass further comprises a stabilizer. The stabilizer is optional and can be butylated hydroxytoluene (BHT), IRGANOX 5057, IRGANOX 245, N-phenyl-2-naphthyl amine, tocotrienol or α-Tocopherol, or any combination thereof. Preferably, the stabilizer is used to stabilize the fluoropolymer additive and hydrophilic polymer (e.g., PVP) in the membrane, particularly during manufacture processes, such as conditioning and E-beam sterilization. In some embodiments, the fluoropolymer additive is SMM1 and the stabilizer is BHT. Other stabilizers having similar antioxidant property are also encompassed herein.

The stabilizer may be mixed with the fluoropolymer additive prior to addition of the fluoropolymer additive to the spin mass, or the stabilizer may be added directly into the spin mass along with the other components thereof (e.g., hydrophobic polymer, hydrophilic polymer, fluoropolymer additive). In embodiments, the spin mass comprises from 0 ppm to 16 ppm, preferably from 2 ppm to 9 ppm, and more preferably from 2 ppm to 7 ppm of a stabilizer. Alternatively, the amount of stabilizer may be expressed in terms of ppm of stabilizer with respect to the fluoropolymer additive (e.g., SMM1). In embodiments, the stabilizer comprises from 0 ppm to 1400 ppm, preferably from 200 ppm to 800 ppm, and more preferably from 200 ppm to 600 ppm with respect to the fluoropolymer additive.

When incorporated into a hollow fiber membrane manufactured according to the present invention, a stabilizer is incorporated in the hollow fiber membrane, helping stabilize the fluoropolymer additive, as determined by one or more techniques, including XPS analysis and molecular weight analysis of the fluoropolymer additive before and after manufacturing. The molecular weight of a fluoropolymer additive, such as SMM1, can be characterized by Gel Permeation Chromatography (GPC). In some embodiments, the average molecular weight of the fluoropolymer additive changes less than 10 wt %, less than 5 wt %, less than 2 wt %, less than 1 wt %, or remains substantially unchanged in the membrane after heating and rinsing during conditioning when the stabilizer is added to the spin mass (based on starting wt % of additive compared to final weight of additive in membrane). In some embodiments, the fluoropolymer additive is more than 65%, more than 75%, more than 85%, or more than 95% retained in the fiber after the fiber spinning process (where the % is determined based on starting wt % of additive compared to final weight of additive in membrane) as determined by thermogravimetric analysis (TGA) of the finished fiber and comparison to the original fluoropolymer additive (e.g., SMM1) concentration in the spin mass.

In embodiments, the method further comprises applying electron beam (E-beam) irradiation to sterilize the resulting filter. E-beam sterilization is widely used in many areas (including the United States) for sterilizing dialyzers for use in hemodialysis and related applications. E-beam sterilization has been suggested as a cause of degradation of the hydrophilic polymers (e.g., PVP) commonly used in such filters. In conventional hollow fiber membrane processes using a polysulfone/PVP blend in the spin mass, a significant amount of the PVP washes out of the formed hollow fiber membranes during rinsing. However, the inventive method combining the use of one or more fluoropolymer additives, such as fluoropolymer-based SMMs, with a hydrophobic base polymer (e.g., PSF), a hydrophilic polymer (e.g., PVP), and a stabilizer, the stabilizer can stabilize the hydrophilic polymer and/or the fluoropolymer additives (e.g., SMM1 and PVP) in the membrane, preventing degradation from conditioning processes and E-beam exposure. Further, as an example, SMM1 is found to bond to PVP. This stabilization effect can be demonstrated using various characterization tests, including by analyzing the molecular weight of SMM1 and the leachable amount of PVP from the E-beam-sterilized dialyzer.

In embodiments, the membrane or dialyzer of the present invention, such as the disclosed SMM1-modified membrane or dialyzer, incorporating one or more stabilizers can be characterized in that the molecular weight of the fluoropolymer additive (e.g., SMM1) is reduced by less than 10%, less than 5%, less than 2%, less than 1%, or is substantially unchanged when conditioned at 100° C. for 6 hours (wherein the % is a wt % and is based on a comparison of the starting wt % and final wt % after 6 hours).

In conventional hollow fiber membrane processes using a polysulfone/PVP blend in the spin mass, a significant amount of the PVP washes out of the formed hollow fiber membranes during rinsing. However, the present invention's manufacturing and post-formation conditioning steps preferably stabilize both the hydrophilic polymer (PVP) and the fluoropolymer additive (e.g., SMM) in the membrane, resulting in a more stable membrane with lower leachability of both agents.

The amount of the hydrophilic polymer (e.g., PVP) leached from the membrane can be tested using simulated extraction condition after E-beam sterilization, with PVP characterized using nuclear magnetic resonance (NMR). In embodiments, the amount of the hydrophilic polymer (e.g., PVP) leached from the membrane (e.g., SMM1-modified membrane) is less than 5%, less than 10%, less than 20%, less than 30%, less than 40%, or less than 50% (based on wt %) that seen from the Standard PSF membrane under identical testing conditions.

In some embodiments, the method produces a membrane with similar hydrophobicity to a conventional membrane but a lower absolute surface charge, leading to reduced platelet adhesion and activation and improved hemocompatibility overall. In some embodiments, the method produces a hemodialysis membrane with improved clearance and/or reduction of middle molecular weight proteins such as beta-2-microglobulin (B2M).

In embodiments, the fluoropolymer additive (e.g., SMM) is added in the spin mass in a concentration sufficient so that at least 5%, at least 6%, at least 7%, at least 8%, at least 9%, or at least 10% (% is atomic %) of the inner surface of the membrane comprises elemental fluorine, as characterized by XPS (F) measurements.

In embodiments, the SMM is SMM1.

In embodiments, the hollow fiber membrane is incorporated into a dialysis filter.

According to another aspect, the present invention relates to a method of using the disclosed hollow fiber membranes and/or dialysis filters to treat a patient.

In embodiments, the treatment involves hemodialysis.

In embodiments, the method does not require the use of therapeutic anticoagulants (e.g., heparin) to prevent clotting. In some embodiments, the method requires less therapeutic anticoagulants than is required without the use of a fluoropolymer additive, such as SMM, for example, 10% less, 20% less, 30% less, 40% less, 50% less, 60% less, 70% less, 80% less, or 90% less (where the % is a wt %).

Membrane Stability.

Zeta Potential. Zeta potential may be used as a marker of membrane stability and hemocompatability. The term “zeta potential ζ” in general is the potential difference between test fluid directed along the surface of a hollow fiber membrane to be examined at a defined velocity. The potential difference between the inlet and the outlet of the test setup is measured. The fluoropolymer additive, such as the surface modifying molecule, renders the surface of the blood contact side of the membrane more hydrophobic and more neutral at the same time. A “higher” or “increased” zeta potential in this case means less negative (closer to neutral) or even more positive, but positive zeta potentials should generally be avoided, because this can cause undesired adsorption of blood proteins and cells that can lead to severe negative effects for the patient. For such a “neutralized” membrane a reduced platelet loss and a reduced TAT III generation is achieved rendering the surface less thrombogenic.

Membrane surface charge is used by some in the industry as a predictor of blood response, particularly hemocompatibility, thrombogenesis and the potential for membrane fouling. Membrane surface characterization demonstrates that hollow fiber membranes incorporating fluoropolymer-based surface modifying molecules have higher (generally speaking, less negative) zeta potentials and greater charge stability after conditioning than conventional membranes after conditioning. This zeta potential effect is expected to be associated with a reduced adsorption of the membrane surface to blood proteins, which in turn is believed to effect measured clearance of various solutes of interest, including middle molecules.

In one aspect of the invention, a membrane of the present invention, such as a charge-stable hollow fiber membrane, maintains a relatively neutral and stable zeta potential around physiological pH. In embodiments, this charge stable or inert membrane is characterized by a zeta potential that is close to neutral and stable with changing pH. In embodiments, this charge stable (or inert) SMM-1 membrane zeta potential is characterized by a slope of zeta potential change vs. pH that is lower than that of a Standard PSF membrane or other conventional membrane or filter. It is understood that in this respect, a “lower” zeta potential vs. pH slope means one that is closer to zero or essentially “flat” when displayed graphically. While tested values for this slope may typically be negative (i.e., displaying a negative slope), it is desirable to avoid highly positive slopes as well. Thus, it is appropriate to describe the slope of zeta potential vs. pH in terms of absolute values of the slopes. In Standard PSF membranes or other conventional membranes, the absolute value of this slope is relatively large, indicating an active membrane surface that is prone to frequent protonation and deprotonation and therefore to more non-specific binding.

In some embodiments, the zeta potential of the membrane of the present invention, such as the SMM1-modified membrane, is greater than −3.5, greater than −3.0, greater than −2.5, or greater than −2.0 when measured at pH 7.5. In some embodiments, the zeta potential of the membrane of the present invention, such as the SMM1-modified membrane is from −3.5 to 0.0, from −3.0 to 0.0, from −2.5 to 0.0, or from −2.0 to 0.0 when measured at pH 7.5.

The relative difference between the zeta potential of a conventional membrane/dialyzer and a modified membrane/dialyzer may indicate the charge-neutralizing effect of modification. The absolute value of this difference in measured zeta potential (ZPD) may be used as a convenient measure or in some cases. For example, where the zeta potential of the Standard PSF membrane (inner luminal surface) is −15.0 mV and the zeta potential of the SMM1-modified membrane is −3.0, the absolute value of the difference in zeta potential (ZPD)=12.0 mV [abs (−15.0 mV −(−3.0 mV)=12.0]. In some cases, a ratio may be taken. Using the same exemplary zeta potential values, a ratio is generated according to: Zeta potential ratio (ZPR)=zeta potential (Standard PSF)/zeta potential (modified)=−15.0 mV/−3.0 mV=5.0. In some embodiments, this zeta potential ratio is greater than 2.0, greater than 2.5, greater than 3.0, greater than 3.5, greater than 4.0, greater than 4.5, or greater than 5.0. It will be understood that a higher ZPD or ZPR indicates a greater degree of passivation of the membrane by the indicated modification.

In some embodiments, the slope of zeta potential vs. pH of the membrane of the present invention, such as the SMM1-modified membrane, is less than −2.00, less than −1.50, less than −1.00, less than −0.8, or less than −0.75. In some embodiments, the slope of zeta potential vs. pH of the membrane of the present invention, such as the SMM1-modified membrane, is from −2.00 to 0.00, from −1.50 to 0.00, from −1.00 to 0.00, from −0.80 to 0.0, or from −0.75 to 0.00. In a particular embodiment, the absolute zeta potential of the membrane of the present invention, such as the SMM1-modified membrane, is from 3.50 to 0.00 when measured at pH 7.5, and the slope of zeta potential vs. pH is from −1.00 to 0.00, according to the measurement methods disclosed herein.

Accordingly, in one aspect, a method of testing dialyzers is disclosed, the method comprising measuring absolute zeta potential across a range of pH values, generating a plot of zeta potential vs. pH, and using the plot to determine the most charge-stable dialyzer, wherein the most charge stable dialyzer is the dialyzer with the most neutral zeta potential at physiological pH and the lowest zeta potential vs. pH slope. In some embodiments, the most charge stable dialyzer is the dialyzer with the most neutral absolute zeta potential and the lowest zeta potential vs. pH slope (expressed as an absolute value). In embodiments, the method is used to identify a dialyzer which has greater hemocompatibility and/or clearance of middle molecules during the course of a dialyzer treatment.

With the present invention, a method to determine the stability of a dialyzer or membrane used in a dialyzer can be utilized. Specifically, in such a method, the dialyzer or membrane used in the dialyzer can be measured for zeta potential (e.g., absolute zeta potential) across at least 2 pH values (e.g., across two, or across three, or across four or more pH values) and then generating a plot of zeta potential vs. pH. In general, the lower the slope, the more stable the dialyzer or membrane used in the dialyzer. Also, with this method, a determination can be made if a test dialyzer or test membrane in a dialyzer is suitable for dialysis with respect to this stability, for instance. The two or more pH values used for the measurement can be any pH value and preferably the pH values are at least 0.5 pH value or at least 1 pH value different from each other (e.g., one pH value is 7.5 and the second pH value is 8 or 8.5 or is 7 or 6.5, for instance). The pH values can be taken from the range of possible pH values and can be taken from a range of a pH of 4 to pH of 8. In this method the zeta potential as described herein is measured for the dialyzer or membrane used in the dialyzer at a first pH value and then a second zeta potential is measured at a second pH value, and optionally at least a third zeta potential is measured at a third pH value, where each pH value is different from each other such that the difference is at least 0.5 or at least 1 for each pH value. If the slope as determined is from −2.00 or less, or −1.5 or less, or −1.0 or less, −0.5 or less, such as −2.00 to 0.00, the dialyzer is considered a charge-stable dialyzer. These values can be absolute values based on an absolute zeta potential value (a slope of 2.00 or less, or 1.5 or less, or 1.0 or less, 0.5 or less, such as 2.00 to 0.00. This method can be based on slope alone or can also take into consideration the zeta potential as described herein. The most charge-stable dialyzer is where the dialyzer has a most neutral zeta potential at physiological pH and the lowest zeta potential vs. pH slope. Further, or alternatively, this method can be used identify or rate a dialyzer with respect to greater hemocompatibility and/or clearance of middle molecules during the course of a dialyzer treatment, as these properties can be present with the desired slope mentioned here.

Contact Angle.

It has been found that a reduced thrombogenicity can be predicted by measuring the contact angle θ of a liquid on the hollow fiber membrane or can be correlated to such measurement. The term “contact angle θ” in general means the angle which is formed between a solid surface in contact with a liquid (in particular water) and the liquid droplet itself and reflects the hydrophobicity/hydrophilicity of the surface. The measurement method for the contact angle θ is described in detail in the Examples section.

In one embodiment, the membrane of the present invention, such as the SMM1-modified membrane, exhibits a contact angle θ of water on the blood contacting surface of the membrane less than 70°, preferably less than 60°, more preferably less than 50°; or a zeta potential (of from −10.0 mV to +5.0 mV, preferably from −6.0 mV to +3.0 mV, more preferably from −4.0 mV to +2.0 mV at physiological pH. Accordingly, in one embodiment, the fluorine-containing hollow fiber membrane exhibits a contact angle θ on its surface of from 60° to 70° and a zeta potential of from −4 mV to +2 mV.

Hemocompatability. Hemocompatability may be measured in a variety of ways, including platelet activation factor 4 (PF4), platelet loss reduction, and TAT III generation.

In embodiments, the present invention relates to a hemocompatible dialysis filter providing for a reduction of platelet loss of at least 10%, at least 20%, at least 30%, at least 40%, at least 50%, at least 60%, at least 70%, at least 80%, or at least 90%, preferably more than 100% when subjected to blood, compared to a dialysis filter that is manufactured identically but without fluoropolymer additive present, the determination of the platelet loss as specified in the description, where the % is based on number or count of platelets.

Middle Molecule Clearance. Significant research indicates that high levels of plasma middle molecular weight proteins may be associated with increased risk of cardiovascular complications in dialysis patients. Accordingly, a goal of the present invention is increased clearance of middle molecules. Lysozyme (MW 14,300) is commonly used as a surrogate for middle molecule clearance. Beta-2-Microglobulin (MW 11,000; B2M) is also used as a reference middle molecule with more direct implications for patient long-term outcomes. Accordingly, dialyzer product literature increasingly reports B2M clearance. High-flux dialysis partly clears middle molecules when used in HD mode, partly by internal filtration, but the hollow fiber membranes of such dialyzers are prone to fouling as proteins build a secondary membrane over the course of the treatment session, reducing potential clearance.

One mechanism to increase middle molecule (e.g., B2M) clearance is to create a more porous (open) membrane than the conventional high flux membranes used in hemodialysis. Manufacturers of so-called medium cut off (MCO) and high cut-off (HCO) membranes report sieving curves (e.g., as determined by dextran sieving before blood contact) with higher molecular weight retention onset (MWRO) and molecular weight cutoff (MWCO) ranges than reported in conventional high flux HD filters. Accordingly, these porous membranes have comparatively high middle molecule clearance. Another approach to increase middle molecule clearance is to use an alternative dialytic mode such as hemodiafiltration (HDF), which drives greater middle molecule clearance by creating convective pressure gradients across a highly permeable membrane. However, both of these approaches are associated with undesirably high loss of blood albumin and are thus not preferred in most chronic ESRD patients. Albumin has a molecular weight of 67,000 Daltons and albumin sieving coefficients are also used to characterize membrane porosity. The instant invention addresses this challenge using membrane processing improvements to more effectively integrate a fluoropolymer additive such as SMM1 into a standard hemodialysis membrane and create a more stable and near charge-neutral blood-contacting surface boundary that is conducive to middle molecule clearance. Accordingly, in some embodiments, a hemodialyzer or membrane suitable for HD therapy is disclosed with high B2M clearance but albumin clearance (sieving coefficient, etc.) comparable to Standard PSF membranes.

The membrane incorporates one or more hydrophobic polymers, one or more hydrophilic polymers, and one or more fluoropolymer additives. The one or more fluoropolymer additives may comprise SMM1.

In embodiments, the B2M clearance is at least 60 ml/min, at least 62 ml/min, at least 65 ml/min, at least 68 ml/min, or at least 70 ml/min, per membrane area of 1.5 m² at a blood flow rate of 300 ml/min, a dialysate flow rate of 500 ml/min, and an ultrafiltration rate of 0.0 ml/min. Clearance data can be measured on hollow fibers of the present invention, for example, according to DIN 58,352. The albumin sieving coefficient can be measured based on ISO8637-1:2017.

In embodiments, the albumin sieving coefficient is less than 0.1, less than 0.05, less than 0.01, less than 0.005, or less than 0.001.

Accordingly, in one embodiment, a dialysis filter is disclosed, where the dialysis filter comprises a plurality of hollow fiber membranes. Each of the hollow fiber membranes comprising: (i) a polysulfone (PSF) base polymer; (ii) a polyvinylpyrrolidone (PVP); and (iii) a fluorine-containing surface modifying macromolecule (SMM) described by formula: F_{T}-[B-(oligo)]n-B-F_{T}, wherein each B comprises a urethane; oligo comprises polypropylene oxide, polyethylene oxide or polytetramethylene oxide; each F_{T} is a polyfluoroorgano group; and n is an integer from 1 to 10, wherein the dialysis filter has a β₂-microglobulin (B2M) clearance of at least 60 ml/min per membrane area of 1.5 m² at a blood flow rate of 300 ml/min, dialysate flow rate of 500 ml/min, and ultrafiltration rate of 0.0 ml/min; and wherein the dialysis filter has an albumin sieving coefficient of less than 0.01.

In embodiments, the fluorine-containing surface modifying macromolecule (SMM) is SMM1.

In embodiments, the albumin sieving coefficient is less than 0.001.

Reduced Secondary Membrane Formation.

Protein adsorption is a critical early event during the interaction of blood with biomaterials. This protein adsorption, in part, triggers subsequent biological responses, including contact activation, the intrinsic coagulation cascade, platelet adhesion, and eventual thrombus formation on biomaterial surfaces. The membrane of the present invention, such as a SMM1-modified dialyzer membrane, reduces the absolute value of the surface charge, which results in lowered protein adsorption. In return, platelet adhesion and platelet activation are reduced. This hypothesis has been demonstrated in in vitro hemocompatibility testing that showed that the platelet adhesion and platelet activation for the SMM1-modified dialyzer were significantly lower compared to the Standard PSF dialyzer.

Accordingly, in some embodiments, the membrane of the present invention, such as a SMM1-modified dialyzer membrane, has a reduced formation of secondary membrane compared to a Standard PSF dialyzer.

### EXAMPLES

### Example 1—Hollow Fiber Membrane Formation

A fluoropolymer-containing hollow fiber membrane was manufactured according to the present invention. A polymer spin mass was prepared using 16.00% by weight of the hydrophobic POLYMER POLYSULFONE (P3500 FROM SOLVAY), 4.00% BY WEIGHT OF THE HYDROPHILIC POLYMER polyvinylpyrrolidone (K81/86 from Ashland), and 0.9% to 1.3% by weight of SMM1 (Interface Biologics, Toronto, CA) based on total weight of the spin mass. BHT was added as a stabilizer to 4.5 ppm in the spin mass. The polymer mixture was filled to 100% with dimethylacetamide (DMAC). SMM1 was prepared according to U.S. Pat. No. 9,884,146 (Compound VII-a) using 1H,1H,2H,2H-perfluorooctanol, hexamethylene diisocyanate and polypropylene oxide as starting materials.

The spin mass was heated to a final temperature of 65-80° C. and degassed so as to produce a homogeneous spinning solution (spin mass). The spin mass was co-extruded through an annular spinneret (tube-in-tube) with a centrally controlled precipitation fluid consisting of 50 wt % DMAC and 50 wt % water by weight. The ratio of DMAC and water can be adjusted within roughly 10% in either direction to meet specifications for membrane permeability. The temperature of the annular spinneret was 38-42° C. The extruded strand was guided through a precipitation gap, of 600 mm. The strand was introduced into a precipitation bath containing 100% water temperature-controlled at 60-70° C. where it solidified into a hollow fiber membrane. The hollow fiber membrane was then routed through rinsing baths temperature-controlled at a temperature of 75° C. to 90° C. The hollow fiber membrane then underwent a drying process between 100° C. and 150° C. The hollow fiber membrane obtained was crimped at a wavelength of approximately 3-5 mm and thereafter taken up on a coiler and formed into a fiber bundle. Each fiber bundle consisted of 11,520 fibers and the final surface of the filters was 1.5 m². Bundles were inserted into OPTIFLUX polycarbonate dialyzer housings and potted using polyurethane according to methods known in the art.

Prior to sterilization, the dialyzers were further rinsed and conditioned according to Table 1 below.

TABLE 1
Conditioning Steps.

| Program | Media | Description | |
|---|---|---|---|
| Conditioning | Saturated | 119-124° | C. |
| | steam | | |
| Rinsing | Water | 65-73° | C. |
| Drying | Air | 104-107° | C. |

Terminal electron-beam (E-beam) sterilization was performed using standard conditions, with filters each receiving between 25 kGy and 55 kGy of radiation. The resulting assembled dialyzer was referred to as the “SMM1-modified dialyzer”. For the control “Standard PSF Dialyzer,” the same manufacturing conditions were used, except SMM1 and BHT were not added to the spin mass. Standard PSF dialyzers and SMM1-modified dialyzers were not steam sterilized, unless specifically noted.

To explore whether this new dialyzer can be incorporated into a heparin-sparing hemodialysis system, a study was designed to evaluate surface characterization looking at membrane microscopic structure, hydrophobicity, elemental analysis, zeta potential, as well as performance and hemocompatibility.

### Example 2—Surface Characterization

During HD, the inner lumen of hemodialyzer hollow fibers come into direct contact with blood; thus, the inner luminal surface of the membrane of SMM1-modified dialyzers was characterized and compared to the membrane of a Standard PSF dialyzer.

Scanning Electron Microscopy (SEM).

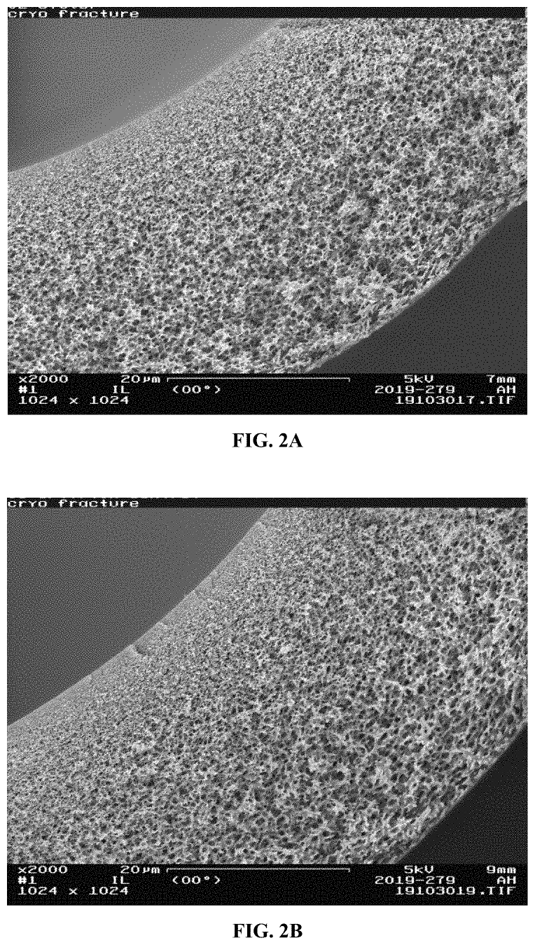

Membrane microscopic structure was evaluated using scanning electron microscopy (SEM). A JSM-6010LA scanning electron microscope (SEM, JEOL, Massachusetts, USA) was used to obtain cross-sectional images of the porous structure of the SMM1-modified membrane and the Standard PSF membrane. Fiber samples were collected from final finished dialyzers and freeze-fractured to preserve the porous structure. Freeze-fracturing involved soaking the fibers in n-hexane, followed by freezing in liquid nitrogen. The frozen fibers were immediately cracked to break and open the fiber cross-section. Fibers were then coated with carbon using a spatter coater for SEM analysis. FIGS. 2A and 2B show scanning electron microscopy (SEM) images of the cross-sectional porous structure of the SMM1-modified membrane (2A) and a Standard PSF membrane (2B).

The membrane porosity morphology of the SMM1-modified membranes has a typical asymmetric structure similar to the morphology of the Standard PSF membranes, which morphology is composed of a densified inner surface and a highly porous outer surface with interconnected pores. The dense inner layer was approximately 1 μm in thickness and the effective pore size radius range of the inner layer was determined to be ˜10-50 Å for both membranes. The addition of SMM1 to the membrane thus did not appear to change the porous structure of the membrane and the asymmetric porous structure was maintained.

Contact Angle.

Contact angle measurements and X-ray photoelectron spectrometry (XPS) were used to characterize incorporation of the fluoropolymer on the inner surface of hollow fiber membranes.

Contact angle was first measured to evaluate the hydrophobicity or hydrophilicity of the inner luminal surface of the SMM1-modified membrane compared to the Standard PSF membrane surface. Measurements were taken using the contact angle system of OCA 15 Plus Goniometry (DataPhysics Instruments USA Corp, North Carolina, USA). Fibers were first separated from the dialyzer and the fiber sample was cut open along the length of the fiber. The cut fiber sample was attached to a glass slide using double-sided tape, with the inner lumen facing up. A 2 μL water droplet was dispensed from a dosing needle and placed onto the membrane surface. The water drop was allowed to sit for 10 seconds. The contact angle of the water drop on the membrane surface substrate was measured using a video-based optical contact angle measuring system using Software SCA 20 (DataPhysics Instruments USA Corp, North Carolina, USA).

The contact angle of the inner lumen of the SMM1-modified membrane (68°±3°) was significantly higher and thus more hydrophobic than the Standard PSF membrane (41.6°±6°) (p<0.05), but the SMM1-modified membrane still maintained surface hydrophilicity (i.e., less than 90°). See Table 2 below. This hydrophilicity of the SMM1-modified membrane allows excess fluid and toxin removal during hemodialysis.

X-Ray Photoelectron Spectrometry (XPS).

An X-ray photoelectron spectrometer (XPS, Kratos, Manchester, UK) was used to quantify the elemental composition (specifically of fluorine) in the top 10 nm of the inner lumen of the SMM1-modified membrane and Standard PSF membranes. Measurements were taken in the surface science lab at the Nanofab lab (University of Utah, UT, USA). Under ultra-low vacuum, an X-ray was used to excite the surface of the material causing ejection of electrons with specific binding energies characteristic of particular atoms. By measuring these characteristic energies, XPS analysis identifies the chemical elements present on the top 3-30 atomic layers (10-100 Å) of samples. The results are shown in Table 2 below. Mean fluorine concentration on the blood-side surface of the SMM1-modified membrane (n=3) was 7.4±0.4% but was undetectable in the Standard PSF membrane (n=3), confirming expectations. This result, combined with the contact angle testing, indicated that SMM1 was successfully incorporated into the inner lumen surface of the SMM1-modified membrane. XPS data also indicated that all SMM1-modified fibers have surface fluorine to various degrees both in the inner surface (IS) that actually comes in contact with blood during hemodialysis and the outer surface (OS) (data not shown).

### Example 3—Zeta Potential at Neutral pH

The zeta potential of the inner lumen of the SMM1-modified membrane and Standard PSF membrane surfaces were measured to characterize membrane surface charge.

The zeta potential was determined using the streaming potential method at Fresenius Medical Care in Ogden, UT, USA and an apparatus in accordance with the zeta potential measuring device described in PCT/EP2020/051078, entitled “Dialyzer Comprising a Fluorine-Containing Hollow Fiber Membrane”, and filed Jan. 17, 2020. A streaming potential develops whenever an electrolyte solution (e.g., potassium chloride, KCl) flows across a charged membrane surface causing a displacement of mobile counter-ions with respect to the fixed charges on the solid surface. This potential is a function of electrolyte flow rates or pressure drop across the surface that drives the movement of the electrolyte. Potassium chloride (KCl) was used to calibrate the system by measuring the initial conductivity and final conductivity after one hour of circulation. Silver/silver chloride (Ag/AgCl) electrodes were used to measure the conductivity of the sample and recorded every 5 minutes along with temperature and pH.

The zeta potential (ζ) was calculated from the streaming potential measurements using the Helmholtz-Smoluchowski Equation [1]:

$$ζ = \frac{η {Λ}_{o}}{{ɛ}_{o} {ɛ}_{r}} \frac{d {E}_{z}}{d Δ P} \qquad [ 1 ]$$

where:
η is the solution viscosity (N·s/m²),
Λₒ is the solution conductivity (A/V·m)
E_{z} is the streaming potential (mV),
εₒ is the permittivity of free space (A·s/V·m)
εᵣ is the solution dielectric constant
ΔP is the pressure drop (N/m²).

Zeta potential measurements of the blood contacting surface showed that the absolute surface charge at the inner lumen was significantly reduced at neutral pH (−3.3 mV±1.1 mV in SMM1-modified membrane, −15.6 mV±1.0 mV in Standard PSF membrane) (p<0.05). In other words, the SMM1-modified membrane is less negatively charged (closer to neutral) than the Standard PSF membrane control. For this type of application, the more desirable value is closer to neutral.

The difference in zeta potential is likely explained by the efficient migration and retention of SMM1 on the inner luminal surface of the SMM1-modified membrane manufactured according to the invention. Terminal fluorinated groups on SMM1 significantly reduce the negative charge on the membrane surface. This is likely due to the compatibility of SMM1 with the base polymers (PVP and PSF) and the free mobility of the terminal fluorinated groups, which could mask the negatively charged PSF and make the surface more neutral at a neutral pH.

Table 2 summarizes the contact angle, XPS F (atomic %), and zeta potential (neutral pH) of the inner lumen of the SMM1-modified membrane and Standard PSF membrane (sample sizes shown). For surface characterization methods contact angle and zeta potential, statistical analysis was performed with two sample t-test to compare differences between the control and test membranes. Differences were analyzed with a 95% confidence level (α=0.05) using Minitab software. Asterisks indicate where statistically significant differences (p<0.05) were present.

TABLE 2
Surface Characterization of Standard
PSF and SMM1-Modified Membrane.

| | Contact Angle | XPS F | Zeta Potential |
|---|---|---|---|
| Samples | (°) | (atomic %) | (mV) |
| Standard PSF | 41 ± 6 | n/a | −15.6 ± 1.0 |
| Membrane | (n = 13)* | (n = 3) | (n = 4)* |
| SMM1 Modified | 68 ± 3 | 7.4 ± 0.4 | −3.3 ± 1.1 |
| Membrane | (n = 18)* | (n = 3) | (n = 3)* |

### Example 4—Reduced Effect of pH on Zeta Potential for SMM1-Modified Membrane

Standard PSF dialyzers (manufactured according to Example 1) and SMM1-modified and Control dialyzers (manufactured according to Example 1 with variations described in Table 3 below) were tested for zeta potential as a function of pH (range 4.0-8.5) to evaluate charge stability.

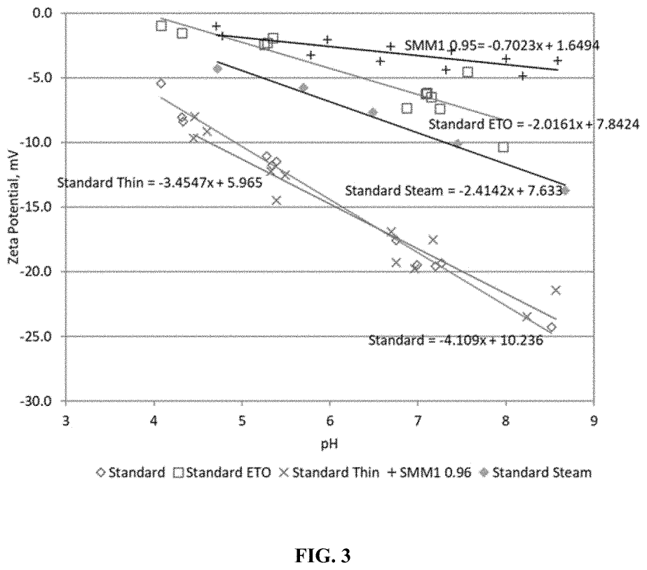

TABLE 3
SMM1-modified and Control Dialyzers
Used in Zeta Potential Testing.

| Dialyzer | Difference Compared |
|---|---|
| (see FIG. 3) | to Example 1 Dialyzers |
| SMM1 0.96 | SMM1-modified dialyzer, SMM1 |
| | content in spin mass at 0.96%. |
| Standard | Standard PSF dialyzer. |
| Standard ETO | Standard PSF dialyzer, but sterilization |
| | changed from E-beam to ethylene oxide (ETO). |
| Standard Steam | Standard PSF dialyzer, but sterilization |
| | changed from E-beam to steam. |
| Standard Thin | Standard PSF dialyzer, but fiber wall |
| | thickness reduced by 15%. |

The test solution was made by dissolving 5.96±0.05 g of potassium chloride (KCL) in 40 L of water, and the pH of the solution was adjusted with KOH and HCL solution of the same molarity as needed. For the Standard Steam control, dialyzers were sterilized using steam as described in PCT/EP2020/051078.

Results are illustrated in FIG. 3. The zeta potential of the E-beam sterilized SMM1 0.96 over the range of 4 pH units varied from −1 to −4 while the zeta potential of the Standard ETO dialyzer varied from −1 to −8. It is apparent from these results that Standard and Standard Thin have similar zeta potential and similar responses to pH change. This trend is as expected because both membranes are manufactured using the same spin mass, similar spinning process, and same post processing conditions.

FIG. 3 further shows that E-beam sterilization has less impact on the surface charge of the SMM1-containing fiber than it does in the other tested dialyzers, but also that the surface charge of the SMM1 modified fiber is more stable across a range of pH values. The slope of the zeta potential vs. pH traces is shown in Table 4 below.

TABLE 4
Slope of Zeta Potential vs. pH.

| Dialyzer | Slone |
|---|---|
| Standard | −4.12 |
| Standard ETO | −2.02 |
| Standard Thin | −3.45 |
| Standard Steam | −2.41 |
| SMM1 0.96 | −0.70 |

FIG. 3 shows the impact of pH on zeta potential for the SMM1-modified dialyzer compared with the Standard PSF dialyzer and control dialyzers. As shown, the pH-zeta potential slope is lowest (nearly neutral/flat) for the SMM1-modified dialyzer. A charge stable filter is expected to demonstrate more efficient clearance over the course of a typical dialysis treatment session because it indicates that the surface is inert to non-specific binding that causes fouling and a deterioration of membrane permeability to diffusible molecules, particularly middle molecules. The greater surface charge stability over a wide range of pH values demonstrates that the SMM1-modified dialyzer is superior to other dialyzers and is predicted to have better hemocompatibility and potentially better middle molecule clearance compared to a comparable dialyzer without the fluoropolymer additive.

### Example 5—Hemocompatability Characterization

In-vitro hemocompatibility of the SMM-1 modified membrane was tested using a variety of indicators and compared to the Standard PSF membrane.

Exaggerated in vitro Thrombosis by Biomarker Analysis. Fresh, healthy, heparinized human donor blood was collected within two hours of testing. The SMM1-modified dialyzer (test dialyzer OPTIFLUX ENEXA F500) and the Standard PSF dialyzer (comparative control dialyzer OPTIFLUX F160NRe) were evaluated for hemocompatibility biomarkers. Both test and control dialyzers had the same membrane surface area of 1.5 m² and a comparable blood compartment volume (85 mL for test, 87 mL for control).

The size-matched test and control dialyzers were first primed by filling the dialysate side of the dialyzers with saline and then filling the patient blood side of the dialyzers with saline. Saline was allowed to recirculate for 10 minutes and then flushed through each dialyzer. After both the test and control dialyzers were completely primed, the donor blood was introduced into the system. Each bag of 500 mL fresh donated blood was used to fill one test dialyzer with 250 mL of blood and one control dialyzer with 250 mL of blood for matched comparisons. Simulations were performed using a blood flow rate of 100 mL/min for 60 minutes. Due to the small volume of donor blood (250 mL), this simulation method (100 mL/min for 60 minutes) was performed to represent roughly the same blood contact time with the dialyzer surface as a standard hemodialysis session with a patient. Samples were taken for complete blood counts (CBC) and platelet activation at 0, 5, 15, 30, 45, and 60 minutes during the simulation. The platelet activation samples were prepared for plasma by centrifugation and analyzed using Platelet Factor 4 (PF-4) ELISA kits (DPF40) (R&D Systems, Inc., Minnesota, USA). Platelet count reduction was analyzed with whole blood from the CBC using the ADVIA 120 hematology system (Siemens Healthineers, Erlanger, Germany). All values were normalized to the hematocrit measured in whole blood at each individual time point by calculating the platelet value at the time point multiplied by the initial hematocrit divided by the hematocrit at the time point. Hematocrit normalization was performed to account for the variability of the blood residues in the blood circuit observed within each simulation over time. After hematocrit normalization, the platelet count was baseline-corrected to the initial platelet count reading using a percentage difference and transformed to observe the loss over time. Mean and standard deviation of each time point were plotted to observe the differences over the course of the simulations.

In vitro Clotting Time. Fresh healthy citrated donor blood was collected and added to each simulation within two hours of collection in accordance with internal procedures. The SMM1-modified dialyzer (test dialyzer OPTIFLUX ENEXA F500) and the Standard PSF dialyzer (comparator control dialyzer OPTIFLUX F160NRe) were evaluated for clotting time and the results were compared. Dialyzers were first primed and filled in the same process as described above. Simulations were performed using a blood flow rate of 300 mL/min for 240 minutes. The blood was recirculated through both the control and test dialyzer circuit and calcium chloride (CaCl₂) was slowly added to both systems simultaneously. The addition of the CaCl₂) forced the blood to slowly clot. The clotting time of each dialyzer was recorded to determine the overall clotting time differences seen between the test and control dialyzers. Clotting was determined by a spike in pressure change. Once a dialyzer clotted, the CaCl₂) additions were stopped for the clotted dialyzer. If a dialyzer did not clot, the simulation was stopped at 240 minutes.

Statistical Analysis. For in vitro thrombosis characterization a mixed effect, repeated measures, two-way ANOVA model was applied, since the in vitro experimental design was an approach with repeated measures and paired samples. Average values and standard deviation (SD) values were calculated from each simulation, then those means were averaged for an overall mean of each group (control and test). Graphpad Prism software 8.4.3 was used for these analyses. Data was analyzed for normality before the parametric tests were determined to be the correct analytical approach. Individual time point differences were analyzed by paired t-tests with a 95% confidence level (α=0.05) using Minitab software. The statistical analysis is displayed on the graphs of the biomarkers as indicated by a p value in the upper left hand corner of the graph and the asterisk above statistically significant time points. The p value listed is reference to the ANOVA statistic and the asterisk reference the paired two tailed t-tests. This analysis approach allows identification of differences between dialyzers and tendencies over time, if any.

For clotting time characterization, data was analyzed for normality before the parametric test was determined to be the correct analytical approach. The differences were analyzed by paired, two tailed t-tests with a 95% confidence level (α=0.05) using Graphpad Prism software 8.4.3. The statistical analysis is displayed on the graph of the clotting times as indicated by a p value in the upper left hand corner of the graph.

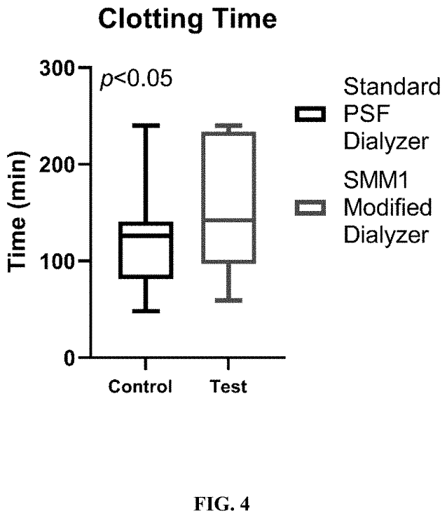

Clotting time evaluations of SMM1-modified dialyzer (n=16) and the Standard PSF dialyzer (n=16) are presented in FIG. 4, illustrating a box-whisker plot of the results. Clotting times were 154.3±65.61 (mean and SD) minutes for the SMM1-modified dialyzer (right) and 129.5±59.60 (mean and SD) minutes for the Standard PSF dialyzer (left), which was significantly different (p<0.05).

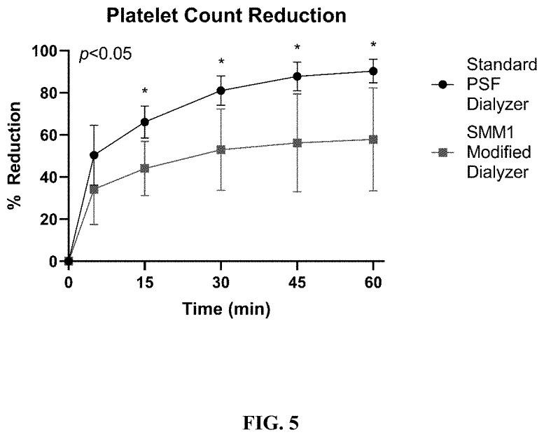

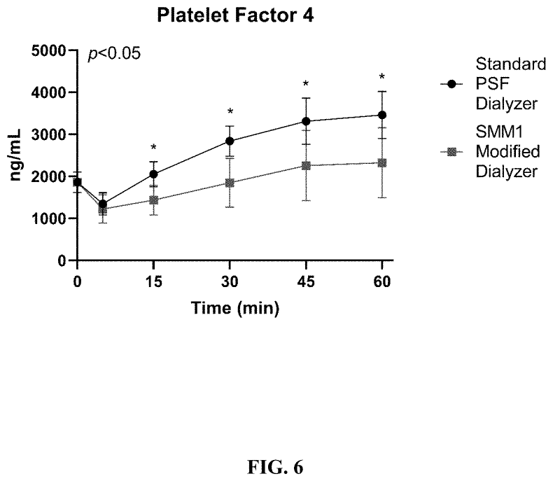

The SMM1-modified dialyzer (n=7) and the Standard PSF dialyzer (n=7) significantly differed in platelet count reduction and platelet activation measured by PF-4 as shown in FIGS. 5 and 6, respectively. The figures display the mean and SD of the simulations at each time point and a connecting line to show the curve over time. The percent platelet count reduction FIG. 5 shows that both the SMM1-modified dialyzer and the Standard PSF dialyzer lose platelets over the course of the experiment, with the Standard PSF dialyzer having more platelet count reduction than the SMM1-modified dialyzer. For platelet count reduction percentage, the Standard PSF dialyzer (62.62%±34.13%) was significantly different from the SMM1-modified dialyzer (40.88%±21.89%) using ANOVA (p<0.05) and significantly different (p<0.05) at time points 15, 30, 45, and 60 minutes using t-test. The platelet activation (PF-4) FIG. 6 shows that both the SMM1-modified dialyzer and the Standard PSF dialyzer have increased concentration of PF-4 (ng/mL) over the course of the experiment with the Standard PSF dialyzer having a higher concentration than the SMM1-modified dialyzer. For platelet activation, the Standard PSF dialyzer (2479.00 ng/mL±852.96 ng/mL) was significantly different from the SMM1-modified dialyzer (1824.10 ng/mL±436.26 ng/mL) using ANOVA (p<0.05) and significantly different (p<0.05) at time points 15, 30, 45, and 60 minutes using t-test.

### Example 6—Stability and Leachability

The SMM1-modified membrane of Example 1 was analyzed to determine how much SMM1 is lost between the spin mass and the final conditioned and E-beam sterilized fiber. Using thermogravimetric analysis (TGA), the SMM1 concentration of the SMM1-modified (finished) membrane was determined to be 4.8%, representing approximately a 25% loss of SMM1 during the manufacturing process. This relatively low loss further demonstrates the stabilizing effect of the inventive method on the fluoropolymer additive and membrane generally.

The stabilizer can prevent degradation of SMM1 during conditioning and/or E-beam sterilization. A stability heating study was performed to simulate the effects of conditioning on the SMM1 material with and without addition of the stabilizer BHT (270 ppm with respect to SMM1). The material was heated at 100° C. continuously for 6 hours, and the molecular weight (MW) of SMM1 was evaluated using GPC. Table 5 illustrates the molecular weight of SMM1 with and without BHT post simulated conditioning study.

TABLE 5
Effect of Stabilizer on SMM1 Degradation (MW) in Conditioning.

| Timing of | Molecular Weight (Da) | |
|---|---|---|
| Heating @100° C. | SMM1 with 270 ppm BHT | SMM1 with no BHT |
| T = 0 | 8095 | 8243 |
| 2 hr | 8254 | 6816 |
| 4 hr | 8086 | 5013 |
| 6 hr | 8155 | N/A |

The results demonstrate that BHT effectively prevents SMM1 degradation from heat conditioning when added to SMM1 that is incorporated into the disclosed SMM1-modified membrane. The use of a stabilizer also reduces overall loss of SMM1 from the membrane following conditioning and E-beam sterilization. Thermogravimetric analysis (TGA).

The stabilizer can also stabilize PVP in the membrane and prevent degradation of PVP to small MW PVP with E-beam exposure, thus preventing small MW PVP being leached out of the membrane during clinical use. This effect further improves patient's safety on dialysis. Both Standard PSF dialyzers and SMM1 modified dialyzers were extracted using simulated use conditions with 1 L 17.2% Ethanol/Water at 37° C. for 24 hours. During the extraction procedure, the dialysate compartment of the tested dialyzer was filled with 17.2% ethanol solution and held static to prevent loss of leachable compounds. The 1L 17.2% ethanol solution recirculated through the blood compartment. At the end of the extraction period, the recirculated extraction medium was collected and analyzed. PVP was analyzed using NMR. Table 6 illustrates that hollow fiber membranes manufactured according to Example 1 have significantly lower leachability of PVP post-conditioning and post E-beam sterilization.

TABLE 6
Reduced Leachability of PVP in SMM1-modified Dialyzer.

| | Standard | SMM1 |
|---|---|---|
| | PSF Dialyzer | Modified Dialyzer |
| | (n = 3) | (n = 3) |
| PVP leachable | 60 +/− 3.2 | 1.5 +/− 0.7 |
| (mg/dialyzer) | | |

### Example 7. Clinical Performance and B2M Removal Rate

A prospective, sequential, multi-center, open-label study was conducted on ESRD subjects on thrice weekly in-center Hemodialysis (HD) to evaluate safety and effectiveness of the SMM1-modified dialyzer (using the OPTIFLUX ENEXA F500 dialyzer) compared to the Standard PSF dialyzer (using the OPTIFLUX F160NR dialyzer). Eligibility criteria were that adults must have been at least 22 years old, on thrice weekly hemodialysis for at least three months prior to enrollment and have been prescribed with the OPTIFLUX F160NR dialyzer for at least 30 days prior the enrollment. Further, subjects needed to have a baseline spKt/V≥1.2, hemoglobin≥9 g/dL, and platelet count ≥100,000/mm³.

The primary study endpoint was in vivo Ultrafiltration Coefficient (Kuf), with secondary endpoints of urea Reduction Ratio and spKt/V, pre and post albumin levels and β2-microglobulin levels, and the number of adverse events and device-related adverse events. In the OPTIFLUX F160NR Period, 23 subjects received 268 HD treatment sessions. In the OPTIFLUX ENEXA F500 Period, 18 subjects received 664 HD treatment sessions.

The results indicated that the OPTIFLUX ENEXA F500 (i.e., SMM1-modified) dialyzer had comparable urea clearance to the OPTIFLUX F160NR, as shown in Table 7 below.

TABLE 7
Urea Clearance, Performance, and β2M Removal.

| | OPTIFLUX | OPTIFLUX | |
|---|---|---|---|
| | ENEXA F500 | F160NR | |
| Measure | (n = 17) | (n = 17) | |
| Kuf (mL/hr/mmHg) | 16.36 (9.92) | N/A | |
| Urea Reduction Rate (%) | 81.87 (5.91) | 80.81 | (4.33) |
| spKt/V | 2.09 (0.41) | 1.94 | (0.3) |
| β2M removal rate (%) | | | |
| a. with correction | 46.86 (7.16) | 67.73 | (16.32) |
| b. without correction | 39.43 (7.39) | 63.75 | (17.14) |

As indicated in Table 7, the performance of the SMM1-modified dialyzer was generally comparable to that of the OPTIFLUX (Standard PSF) dialyzer and the SMM1-modified dialyzer was well tolerated. Mean urea reduction ratio (82 vs. 81%), and spKt/V (2.1 vs. 1.9) were comparable for the SMM1-modified dialyzers and OPTIFLUX (Standard PSF) dialyzers.

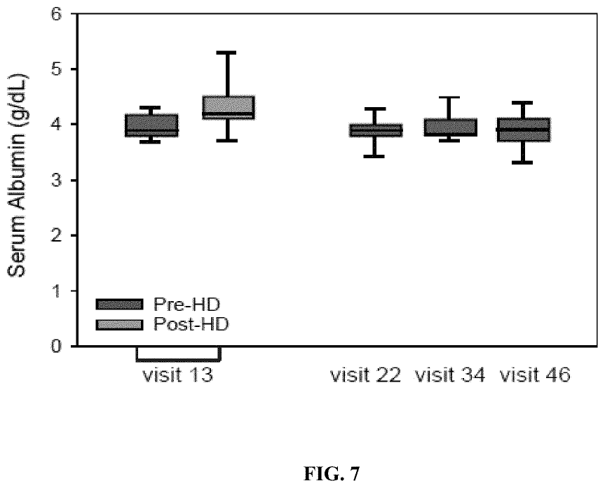

Serum albumin levels were also measured in study participants on the SMM1-modified dialyzer both pre-HD and post-HD (week 13 only). As shown in FIG. 7, pre-HD mean serum albumin levels (week 13, and visits 22, 34, and 46) were comparable for all visits, and pre-HD mean serum albumin level (left box) was comparable to post-HD mean serum albumin level (right box) at week 13. Albumin sieving coefficients were measured in the SMM1-modified dialyzer using methods known to the art. Clearance data can be measured on hollow fibers of the present invention, for example, according to DIN 58,352 or ISO8637-1:2017. The albumin sieving coefficient can be measured based on ISO8637-1:2017 having about 1.5 m² area.

Using bovine plasma protein (60 g/L, 37° C.), at a blood flow rate Qb=300 mL/min and ultrafiltration rate=29 mL/min, the measured albumin sieving coefficient was less than 0.01, comparable to the OPTIFLUX (Standard PSF) dialyzer reference standard.

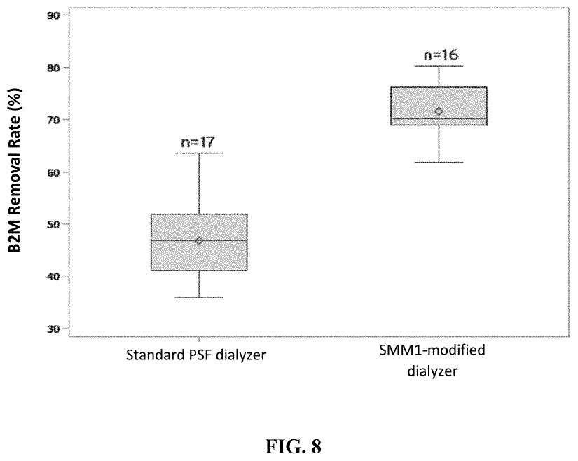

Surprisingly, however, the OPTIFLUX ENEXA dialyzer (n=16) showed a significantly higher β2-microglobulin removal rate than the OPTIFLUX control dialyzer (n=17) in the clinical study. FIG. 8 shows the β2M removal rate from the study. Specifically, the OPTIFLUX ENEXA (SMM1-modified) dialyzer exhibited a 68% β2M removal rate compared to 47% in the Standard PSF dialyzer.

### Example 8—In Vitro Middle Molecule Clearance

The in vitro clearance of a given solute by a hollow fiber membrane is determined according to the hollow fiber membrane filter structured as per DIN ISO 8637-1:2017. Sample dialyzers were prepared for the SMM1-modified dialyzer (OPTIFLUX F160 dialyzer, surface area 1.5 m², n=6) and Standard PSF dialyzer (OPTIFLUX F160NR dialyzer, surface area 1.5 m², n=5). An immunoturbidimetric assay was used for quantitative in vitro determination of β2M in human serum and plasma (Roche/Hitachi systems). Latex-bound anti-β2M antibodies react with antigen from the sample to form antigen/antibody complexes which are determined turbidimetrically after agglutination. The color intensity was directly proportional to the concentration which is determined photometrically at 700 nm.

Clearance is measured using the following formula:

$$K = ( 1 - \frac{{C}_{O}}{{C}_{I}} ) * Qb$$

where K=clearance value; C_{O}=Clearance outlet value; C₁=Clearance inlet value, and Q_{b}=Blood side flow rate (mL/min.). A flow rate of 300 ml/min was set in the blood chamber, a flow rate of 500 ml/min was set in the dialysis chamber, and ultrafiltration rate was set to 0 ml/min. Table 8 shows the average 02M clearance in both dialyzers.

TABLE 8
In vitro β2M Clearance.

| | Average β2M Clearance | Standard Deviation |
|---|---|---|
| Dialyzer | (mL/min) | (mL/min) |
| OPTIFLUX F160NR | 49.15 | 4.75 |
| (Standard PSF dialyzer) | | |
| OPTIFLUX ENEXA | 68.67 | 7.65 |

As shown in Table 8, the in-vitro β2M clearance of the SMM1-modified dialyzer was significantly higher than the control dialyzer (p<0.05).

These results taken together suggest a lower heparin requirement in patients treated using the SMM1-modified dialyzer. A dialyzer with reduced platelet activation and blood clotting could potentially reduce long and short-term complications due to use of anticoagulants, reduce heparin and other anemia-managing pharmaceutical requirements, and improve outcomes in dialysis patients.

### Example 9. Effect of Conditioning and Sterilization on Fluoropolymer Additive Migration

A set of SMM1-modified dialyzers were manufactured according to Example 1 with the SMM1 concentration in the spin mass set at 0.9% (herein referred to as “0.9% SMM1”). Another set of dialyzers was manufactured according to Example 1 with an SMM1 concentration in the spin mass of 0.9%, but without the conditioning and E-beam sterilization steps (herein referred to as “0.9% SMM1-Raw”)

The fluorine (F) content on the inner lumen surface of 0.9% SMM1 and 0.9% SMM1-Raw were evaluated using the XPS method described in Example 2. The results (Table 9) show that the conditioning and E-beam process enriched the F content on the surface of the membrane.

TABLE 9
Effect of Conditioning and E-beam Sterilization on Fluoropolymer
Additive Migration in Hollow Fiber Membranes.

| | F % by XPS | | |
|---|---|---|---|
| | AVG | SD | n |
| 0.9% SMM1 | 6.35 | 0.63 | 6 |
| 0.9% SMM1-Raw | 5.51 | 0.64 | 2 |

In general, the measurement techniques and/or tests and/or dialyzer set up and/or any other details as set forth in US-2020-0188860-A1 can be used here and this publication is incorporated in its entirety by reference herein. Further, the measurement of the albumin sieving coefficient or other membrane properties of the hollow fiber membrane can be carried out on a finished hollow fiber membrane filter according to DIN EN ISO 8637:2014.

The present invention includes the following aspects/embodiments/features in any order and/or in any combination:

1. The present invention relates to a hollow fiber membrane for blood purification, the hollow fiber membrane comprising:

(i) a hydrophobic base polymer;

(ii) a hydrophilic polymer;

(iii) a fluoropolymer additive; and

(iv) optionally a stabilizer,

wherein a fluorine content on an inner surface of the hollow fiber membrane is from 5 to 10 atomic % (F), as determined by X-ray photoelectron spectrometry (XPS).
2. The hollow fiber membrane of any preceding or following embodiment/feature/aspect, wherein the hydrophobic base polymer is a polysulfone (PSF), a polyethersulfone (PES), a poly(aryl) ethersulfone (PAES), or any combinations thereof.
3. The hollow fiber membrane of any preceding or following embodiment/feature/aspect, wherein the hydrophilic polymer comprises a polyvinylpyrrolidone (PVP), a polyethyleneglycol (PEG), or a polypropylene glycol (PPG).
4. The hollow fiber membrane of any preceding or following embodiment/feature/aspect, wherein the fluoropolymer additive comprises a surface-modifying macromolecule having a formula:

F_{T}-[B-(oligo)]n-B-F_{T}

wherein each B comprises a urethane; oligo comprises a polypropylene oxide, a polyethylene oxide or a polytetramethylene oxide;
each F_{T} is a polyfluoroorgano group; and
n is an integer from 1 to 10.
5. The hollow fiber membrane of any preceding or following embodiment/feature/aspect, wherein the stabilizer is present and is a butylated hydroxytoluene (BHT), IRGANOX 5057, IRGANOX 245, N-phenyl-2-naphthyl amine, tocotrienol, α-Tocopherol, or any combinations thereof.
6. The hollow fiber membrane of any preceding or following embodiment/feature/aspect, wherein the hollow fiber membrane comprises from 2 ppm to 7 ppm of the stabilizer.
7. The hollow fiber membrane of any preceding or following embodiment/feature/aspect, wherein the molecular weight of the fluoropolymer additive is reduced by less than 5 wt %, based on the total weight of said fluoropolymer present in said hollow fiber membrane when said hollow fiber membrane is conditioned at 100° C. for 6 hours.
8. The hollow fiber membrane of any preceding or following embodiment/feature/aspect, wherein said F_{T} is 1H,1H,2H,2H-perfluorooctanol, said B is a hexamethylene diisocyanate based urethane, and said oligo is said polypropylene oxide.
9. The hollow fiber membrane of any preceding or following embodiment/feature/aspect, wherein the fluoropolymer additive is a SMM1.
10. The hollow fiber membrane of any preceding or following embodiment/feature/aspect, wherein the fluorine content on the inner surface of the hollow fiber membrane is from 7 to 10 atomic % (F), as measured by X-ray photoelectron spectrometry (XPS).
11. The hollow fiber membrane of any preceding or following embodiment/feature/aspect, wherein the fluoropolymer additive is more than 75 wt % retained in the hollow fiber membrane after formation by a fiber spinning process.
12. The hollow fiber membrane of any preceding or following embodiment/feature/aspect, wherein a zeta potential of the hollow fiber membrane is from −6.0 mV to +3.0 mV at a pH of 7.5.
13. The hollow fiber membrane of any preceding or following embodiment/feature/aspect, wherein a zeta potential of the hollow fiber membrane is from −4.0 mV to +2.0 mV at a pH of 7.5.
14. The hollow fiber membrane of any preceding or following embodiment/feature/aspect, wherein a contact angle θ of the hollow fiber membrane is from 60° to 70° and a zeta potential of the hollow fiber membrane is from −4.0 mV to +2.0 mV at a pH of 7.5.
15. The hollow fiber membrane of any preceding or following embodiment/feature/aspect, wherein a slope of a curve of a zeta potential of the hollow fiber membrane vs pH of zeta potential measurement is less than −2.0.
16. The hollow fiber membrane of any preceding or following embodiment/feature/aspect, wherein a slope of a curve of a zeta potential of the hollow fiber membrane vs pH of zeta potential measurement is less than −1.5.
17. The hollow fiber membrane of any preceding or following embodiment/feature/aspect, wherein a slope of a curve of a zeta potential of the hollow fiber membrane vs pH of zeta potential measurement is less than −1.0.
18. The present invention further relates to a dialysis filter for use in hemodialysis, the dialysis filter comprising the hollow fiber hollow fiber membrane of any preceding or following embodiment/feature/aspect described.
19. The dialysis filter of any preceding or following embodiment/feature/aspect, wherein the hydrophobic base polymer comprises a polysulfone, the hydrophilic polymer comprises a polyvinylpyrrolidone (PVP); and the fluoropolymer additive has a formula:

F_{T}-[B-(oligo)]n-B-F_{T}

wherein each B comprises a urethane;
oligo comprises a polypropylene oxide, a polyethylene oxide or a polytetramethylene oxide;
each F_{T} is a polyfluoroorgano group; and
n is an integer from 1 to 10.

wherein the dialysis filter has a β₂-microglobulin (B2M) clearance of at least 60 ml/min per membrane area of 1.5 m² at a blood flow rate of 300 ml/min, a dialysate flow rate of 500 ml/min, and an ultrafiltration rate of 0.0 ml/min; and

wherein the dialysis filter has an albumin sieving coefficient of less than 0.01.

20. The dialysis filter of any preceding or following embodiment/feature/aspect, wherein the fluoropolymer additive is SMM1.
21. The dialysis filter of any preceding or following embodiment/feature/aspect, wherein the B2M clearance is at least 65 ml/min.
22. The dialysis filter of any preceding or following embodiment/feature/aspect, wherein the B2M clearance is at least 68 ml/min.
23. The dialysis filter of any preceding or following embodiment/feature/aspect, wherein the dialysis filter is a hemodialysis filter.
24. The dialysis filter of any preceding or following embodiment/feature/aspect, wherein the hollow fiber membrane has a zeta potential of from 0.0 mV to −4.0 mV at a pH of 7.5.
25. The dialysis filter of any preceding or following embodiment/feature/aspect, wherein the hollow fiber membrane has a zeta potential of from 0.0 mV to −2.0 mV at a pH of 7.5.
26. The dialysis filter of any preceding or following embodiment/feature/aspect, wherein the hollow fiber membrane has a contact angle of from 50 to 70 degrees.
27. The dialysis filter of any preceding or following embodiment/feature/aspect, wherein the filter is electron-beam (E-beam) sterilized.
28. The dialysis filter of any preceding or following embodiment/feature/aspect, said dialysis filter comprising a plurality of the hollow fiber membrane, wherein said B2M clearance is at least 65 ml/min, and said plurality having a zeta potential of from 0.0 mV to −4.0 mV at a pH of 7.5, and

wherein a slope of a curve of a zeta potential of the plurality of the hollow fiber membrane vs pH of zeta potential measurement is less than −2.0.

29. The dialysis filter of any preceding or following embodiment/feature/aspect, for use in treating a dialysis patient.
30. The dialysis filter of any preceding or following embodiment/feature/aspect, wherein the dialysis filter is electron-beam (E-beam) sterilized.
31. The dialysis filter of any preceding or following embodiment/feature/aspect, wherein the albumin sieving coefficient is less than 0.001.
32. The present invention further relates to a method of manufacturing the dialysis filter of the present invention of any preceding or following embodiment/feature/aspect, the method comprising:

A) preparing a spin mass comprising an aprotic solvent, the hydrophobic base polymer, the hydrophilic polymer, and the fluropolymer additive in a concentration of from 0.9% to 1.3% w/w, based on the total weight of the spin mass;

(B) extruding said spin mass from an outer annular orifice through a tube in-orifice spinneret into an aqueous solution to form the hollow fiber membrane, and

(C) isolating the hollow fiber membrane,

wherein the dialysis filter has a beta-2-microglobulin (B2M) reduction ratio greater than 60%; a β₂-microglobulin (B2M) clearance of at least 65 ml/min per membrane area of 1.5 m² at a blood flow rate of 300 ml/min, a dialysate flow rate of 500 ml/min, and an ultrafiltration rate of 0.0 ml/min; and an albumin sieving coefficient of less than 0.01, when operated in a hemodialysis mode.
33. The method of any preceding or following embodiment/feature/aspect, wherein the hydrophobic base polymer is a polysulfone (PSF), a polyethersulfone (PES) and a poly(aryl) ethersulfone (PAES), and the hydrophilic polymer is a polyvinylpyrrolidone (PVP), a polyethyleneglycol (PEG), a polyvinylalcohol (PVA), or a copolymer of a polypropyleneoxide and a polyethyleneoxide (PPO-PEO).
34. The method of any preceding or following embodiment/feature/aspect, wherein the fluoropolymer additive is more than 75 wt % retained in the hollow fiber membrane after said isolating.
35. The method of any preceding or following embodiment/feature/aspect, wherein the hollow fiber membrane has a zeta potential of from 0.0 mV to −2.0 mV at a pH of 7.5.
36. The method of any preceding or following embodiment/feature/aspect, wherein the hollow fiber membrane has a contact angle of from 50 to 70 degrees.
37. The method of any preceding or following embodiment/feature/aspect, wherein a slope of a curve of a zeta potential of the hollow fiber membrane vs pH of zeta potential measurement is less than −2.0.
38. The method of any preceding or following embodiment/feature/aspect, wherein the spin mass is heated to a temperature of 65-80° C.; and said extruding is with a centrally-controlled precipitation fluid consisting of a mixture of diemethylacetamide (DMAC) and water; and after said isolating, conditioning the hollow fiber membrane by exposing the hollow fiber membrane to saturated steam, and then rinsing with water, and then air drying prior to a sterilization.
39. The method of any preceding or following embodiment/feature/aspect, wherein the stabilizer is a butylated hydroxytoluene (BHT), IRGANOX 5057, IRGANOX 245, N-phenyl-2-naphthyl amine, tocotrienol, α-Tocopherol, and a combination of any thereof.
40. The method of any preceding or following embodiment/feature/aspect, wherein the spin mass comprises from 2 ppm to 7 ppm of the stabilizer.
41. The method of any preceding or following embodiment/feature/aspect, wherein the spin mass is heated to 75-80° C.
42. The method of any preceding or following embodiment/feature/aspect, wherein the centrally-controlled precipitation fluid consists of 50 wt % DMAC and 50 wt % water by total weight of the centrally-controlled precipitation fluid.
43. The method of any preceding or following embodiment/feature/aspect, wherein a temperature of the tube in-orifice spinneret is maintained at 35-45° C. during said extruding.
44. The method of any preceding or following embodiment/feature/aspect, wherein the temperature of the tube in-orifice spinneret is maintained at 38-42° C. during said extruding.
45. The method of any preceding or following embodiment/feature/aspect, wherein after said extruding, the hollow fiber membrane is guided through a precipitation gap of 200-600 mm prior to introduction into a precipitation bath.
46. The method of any preceding or following embodiment/feature/aspect, wherein the precipitation gap is about 600 mm.
47. The method of any preceding or following embodiment/feature/aspect, wherein the fluoropolymer additive is SMM1.
48. The method of any preceding or following embodiment/feature/aspect, wherein the SMM1 is added to the spin mass in a concentration of from 0.4 wt % to 1.9 wt % SMM1, based on total weight of the spin mass.
49. The method of any preceding or following embodiment/feature/aspect, wherein the SMM1 is added to the spin mass in a concentration of from 0.8 wt % to 1.6 wt %, based on total weight of the spin mass.
50. The method of any preceding or following embodiment/feature/aspect, wherein the SMM1 is added to the spin mass in a concentration of from 0.9 wt % to 1.3 wt %, based on total weight of the spin mass.
51. The present invention also relates to a method of hemodialysis, the method comprising passing a blood through a first chamber of the dialysis filter of the present invention based on any preceding or following embodiment/feature/aspect, such that the blood contacts a first side of the hollow fiber membrane; and passing a dialysis solution through a second chamber of the dialysis filter such that the dialysis solution contacts a second opposite side of the membrane, such as a porous asymmetric membrane, to remove waste products from the blood, wherein the first chamber is inside the hollow fiber membrane and the second chamber is between the hollow fiber membrane and an inner wall of the dialysis filter.

The present invention can include any combination of these various features or embodiments above and/or below as set forth in sentences and/or paragraphs. Any combination of disclosed features herein is considered part of the present invention and no limitation is intended with respect to combinable features.

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

Other embodiments of the present invention will be apparent to those skilled in the art from consideration of the present specification and practice of the present invention disclosed herein. It is intended that the present specification and examples be considered as exemplary only with a true scope and spirit of the invention being indicated by the following claims and equivalents thereof.

As used herein, an element or operation recited in the singular and proceeded with the word “a” or “an” should be understood as not excluding plural elements or operations, unless such exclusion is explicitly recited. In other words, “a” or “an” include one or at least one or more than one. Furthermore, references in the present disclosure to “one embodiment”, “an embodiment”, or even “a preferred embodiment” are not intended to exclude additional embodiments that also incorporate the recited features.

Although specific embodiments have been illustrated and described herein, it should be appreciated that any arrangement calculated to achieve the same purpose may be substituted for the specific embodiments shown. This disclosure is intended to cover any and all adaptations or variations of various embodiments. It is to be understood that the above description has been made in an illustrative fashion, and not a restrictive one. Combinations of the above embodiments, and other embodiments not specifically described herein will be apparent to those of skill in the art upon reviewing the above description. Thus, the scope of various embodiments includes any other applications in which the above compositions, structures, and methods are used. Accordingly, the claims set forth below should be construed in view of the full breadth and spirit of the present invention as described herein.

1. A hollow fiber membrane for blood purification, the hollow fiber membrane comprising:
(i) a hydrophobic base polymer;
(ii) a hydrophilic polymer;
(iii) a fluoropolymer additive; and
(iv) optionally a stabilizer,
wherein a fluorine content on an inner surface of the hollow fiber membrane is from 5 to 10 atomic % (F), as determined by X-ray photoelectron spectrometry (XPS).

2. (canceled)

3. (canceled)

4. The hollow fiber membrane of claim 1, wherein the fluoropolymer additive comprises a surface-modifying macromolecule having a formula:
F_{T}-[B-(oligo)]n-B-F_{T}
wherein each B comprises a urethane; oligo comprises a polypropylene oxide, a polyethylene oxide or a polytetramethylene oxide;
each F_{T} is a polyfluoroorgano group; and
n is an integer from 1 to 10.

5. The hollow fiber membrane of claim 1, wherein the stabilizer is present and is a butylated hydroxytoluene (BHT), IRGANOX 5057, IRGANOX 245, N-phenyl-2-naphthyl amine, tocotrienol, α-Tocopherol, or any combinations thereof.

6. The hollow fiber membrane of claim 1, wherein the hollow fiber membrane comprises from 2 ppm to 7 ppm of the stabilizer.

7. (canceled)

8. (canceled)

9. The hollow fiber membrane of claim 4, wherein the fluoropolymer additive is a SMM1.

10. The hollow fiber membrane of claim 1, wherein the fluorine content on the inner surface of the hollow fiber membrane is from 7 to 10 atomic % (F), as measured by X-ray photoelectron spectrometry (XPS).

11. (canceled)

12. The hollow fiber membrane of claim 1, wherein a zeta potential of the hollow fiber membrane is from −6.0 mV to +3.0 mV at a pH of 7.5.

13. (canceled)

14. The hollow fiber membrane of claim 1, wherein a contact angle θ of the hollow fiber membrane is from 60° to 70° and a zeta potential of the hollow fiber membrane is from −4.0 mV to +2.0 mV at a pH of 7.5.

15. The hollow fiber membrane of claim 1, wherein a slope of a curve of a zeta potential of the hollow fiber membrane vs pH of zeta potential measurement is less than −2.0.

16. (canceled)

17. (canceled)

18. A dialysis filter for use in hemodialysis, the dialysis filter comprising the hollow fiber hollow fiber membrane of claim 1.

19. The dialysis filter of claim 18, wherein the hydrophobic base polymer comprises a polysulfone, the hydrophilic polymer comprises a polyvinylpyrrolidone (PVP); and the fluoropolymer additive has a formula:
F_{T}-[B-(oligo)]n-B-F_{T}
wherein each B comprises a urethane;
oligo comprises a polypropylene oxide, a polyethylene oxide or a polytetramethylene oxide;
each F_{T} is a polyfluoroorgano group; and
n is an integer from 1 to 10.
wherein the dialysis filter has a β₂-microglobulin (B2M) clearance of at least 60 ml/min per membrane area of 1.5 m² at a blood flow rate of 300 ml/min, a dialysate flow rate of 500 ml/min, and an ultrafiltration rate of 0.0 ml/min; and
wherein the dialysis filter has an albumin sieving coefficient of less than 0.01.

20. The dialysis filter of claim 19, wherein the fluoropolymer additive is SMM1.

21. (canceled)

22. (canceled)

23. The dialysis filter of claim 19, wherein the dialysis filter is a hemodialysis filter.

24. The dialysis filter of claim 19, wherein the hollow fiber membrane has a zeta potential of from 0.0 mV to −4.0 mV at a pH of 7.5.

25. (canceled)

26. The dialysis filter of claim 25, wherein the hollow fiber membrane has a contact angle of from 50 to 70 degrees.

27. (canceled)

28. The dialysis filter of claim 20, said dialysis filter comprising a plurality of the hollow fiber membrane, wherein said B2M clearance is at least 65 ml/min, and said plurality having a zeta potential of from 0.0 mV to −4.0 mV at a pH of 7.5, and
wherein a slope of a curve of a zeta potential of the plurality of the hollow fiber membrane vs pH of zeta potential measurement is less than −2.0.

29. (canceled)

30. (canceled)

31. (canceled)

32. A method of manufacturing the dialysis filter of claim 18, the method comprising:
A) preparing a spin mass comprising an aprotic solvent, the hydrophobic base polymer, the hydrophilic polymer, and the fluropolymer additive in a concentration of from 0.9% to 1.3% w/w, based on the total weight of the spin mass;
(B) extruding said spin mass from an outer annular orifice through a tube in-orifice spinneret into an aqueous solution to form the hollow fiber membrane, and
(C) isolating the hollow fiber membrane,
wherein the dialysis filter has a beta-2-microglobulin (B2M) reduction ratio greater than 60%; a β₂-microglobulin (B2M) clearance of at least 65 ml/min per membrane area of 1.5 m² at a blood flow rate of 300 ml/min, a dialysate flow rate of 500 ml/min, and an ultrafiltration rate of 0.0 ml/min; and an albumin sieving coefficient of less than 0.01, when operated in a hemodialysis mode.

33.-37. (canceled)

38. The method of claim 32, wherein the spin mass is heated to a temperature of 65-80° C.; and said extruding is with a centrally-controlled precipitation fluid consisting of a mixture of diemethylacetamide (DMAC) and water; and after said isolating, conditioning the hollow fiber membrane by exposing the hollow fiber membrane to saturated steam, and then rinsing with water, and then air drying prior to a sterilization.

39.-42. (canceled)

43. The method of claim 38, wherein a temperature of the tube in-orifice spinneret is maintained at 35-45° C. during said extruding.

44.-46. (canceled)

47. The method of claim 38, wherein the fluoropolymer additive is SMM1.

48. The method of claim 47, wherein the SMM1 is added to the spin mass in a concentration of from 0.4 wt % to 1.9 wt % SMM1, based on total weight of the spin mass.

49.-51. (canceled)
